(19) United States
(12) Patent Application Publication
(10) Pub. No.: US 2018/0346960 A1
(43) Pub. Date: Dec. 6, 2018

(54) Fluorogenic peptide substrates for in solution and solid phase factor Xa measurements

(71) Applicant: Eugene Y. Chan, Boston, MA (US)

(72) Inventor: Eugene Y. Chan, Boston, MA (US)

(21) Appl. No.: 15/993,442

(22) Filed: May 30, 2018

Related U.S. Application Data
(60) Provisional application No. 62/512,443, filed on May 30, 2017.

(51) Int. Cl. C12Q 1/37; C07K 1/04

(52) U.S. Cl. CPC C12Q 1/37; C07K 1/047

(57) ABSTRACT

The measurement of Factor Xa (FXa) enzymatic activity using novel fluorogenic peptide substrates that have a C-terminus cleavable fluorophore and optionally the ability to attach to a solid support. Fluorogenic measurements increase sensitivity and flexibility of measurements of enzymatic reactions over traditional absorbance-based approaches. The measurement of FXa generation is applicable to a range of biological reactions.

### CROSS-REFERENCE TO RELATED APPLICATIONS

The present application claims the benefit of co-pending U.S. provisional application No. 62/512,443, filed on May 30, 2017, the entire disclosure of which is incorporated by reference as if set forth in its entirety herein.

### TECHNICAL FIELD

Embodiments described herein generally relate to the measurement of Factor Xa (FXa) enzymatic activity using novel fluorogenic peptide substrates that have a C-terminus cleavable fluorophore. Some embodiments relate to peptides linked to solid supports. In some embodiments, fluorogenic measurements increase sensitivity and flexibility of measurements of enzymatic reactions over traditional absorbance-based approaches. The measurement of FXa generation may be applicable to a range of biological reactions. In some embodiments, the assay may be implemented in solution or on solid support.

### BACKGROUND

Blood coagulation cascades are initiated by extrinsic and intrinsic pathways. Both pathways lead to the activation of Factor X (FX) into Factor Xa (FXa), which in combination with activated Factor V (FVa) on cell surface membranes with Ca²⁺ further activates prothrombin into thrombin to cause blood clotting. The extrinsic pathway is activated by damaged blood vessels, from which tissue factor (TF) is released, leading to the activation of Factor VII (FVII). FIX and FX are converted to FIXa and FXa, respectively by the TF-FVIIa complex. The intrinsic pathway is initiated when blood is exposed to foreign surface. This process requires the assembly of Factor IXa (FIXa) and Factor VIII (FVIII) on lipid membrane with the present of Ca²⁺ to activate FX into FXa.

FVIII and FIX are significant molecules in the coagulation cascade. FVIII and FIX are produced by FVIII and FIX genes located in the X chromosome. Mutations in these genes may cause hemophilia, which occurs 1/5000 live male births. FVIII deficiency is responsible for hemophilia A and FIX deficiency is responsible for hemophilia B. Individuals with plasma factor levels less than 1%, between 2-5%, and between 6%-40% correspond to severe, moderate, and mild forms of hemophilia, respectively.

Hemophilia is treated with factor replacement therapy. The first type of treatment is plasma-derived products which are extracted and manufactured from pooled human plasma into patients' circulatory systems. Newer therapies are based on recombinant factors derived from Chinese hamster ovary (CHO), baby hamster kidney (BHK) and human embryonic kidney (HEK) cells. Particular human factor genes are injected into these cells to produce large amounts of recombinant protein. Because the factors are produced in mammalian cells, infections from human pathogens such as HIV are largely reduced. Long-acting versions of FVIII and FIX are based on PEGylation or recombinant Fc or albumin fusion proteins, allowing for reduction in injection frequency.

Therapeutic dosing of replacement factors is important since levels may vary for a patient depending on a variety of patient-specific variables. Frequent measurement of drug levels will decrease the risk for spontaneous bleeding and side effects, ultimately increasing patient compliance, safety, and health.

There are several methodologies available to test the FVIII activity in humans. Today, these include predominantly the one-stage clotting assay and the chromogenic assay. The one-stage assay is based on activated partial thromboplastin time (APTT) test. Patient plasma is added to FVIII deficient plasma to test the APTT. Patient plasma APTT result is compared to the standard curve to identify any abnormalities. The two-stage clotting assay improved upon the one-stage assay and is best embodied by the chromogenic assay. In the first stage, the sample is diluted into a buffer that includes reagents to generate FXa. DIE is activated by trace amounts of thrombin and then becomes the rate-determining reagent with other factors (FIXa, FX) provided in excess. The second stage involves the cleavage of a chromogenic substrate by FXa to determine the amount of FXa generated as a function of the FVIII starting concentration. Similarly, FIX can be measured by activation with FXIa and providing excess FVIIIa and FX to produce FXa.

It is commonly recognized that the chromogenic assay is the most accurate method in current commercial market for measurement of FXa. The chromogenic substrate commonly used in commercial FVIII/FIX assays is para-nitroaniline (pNA) substrate. This type of substrate has FXa cleavage site linked to the pNA molecule. After cleavage, chromogenic pNA will be released into the solution. The signal can be measured using optical absorption.

There are several limitations of the pNA substrate. Firstly, the sensitivity level of pNA substrate is low. The definition of severe and mild hemophilia is 1% and 2-5%, respectively. The detection of low FVIII level with pNA substrate may not be very accurate. Two assay ranges, a high and a low, are typically required to span the desired measurements. Secondly, due to the chemical structure, pNA molecule is monofunctional, which means the molecule can only have one site linked to other chemicals. Therefore, the application of the pNA substrate is limited to solution phase only. Thirdly, the wavelength of spectrophotometer detection of pNA is very similar to hemoglobin. Contamination of hemoglobin in plasma may adversely affect the results.

Existing fluorogenic substrates for FXa have limitations in sensitivity, specificity, and chemistry functionality. For instance, there is a commercially available CH₃SO₂-D-CHA-Gly-Arg-AMC sequence that is not as sensitive and specific for FXa. Furthermore, upon cleavage, the AMC fluorophore cannot be linked to the solid-phase.

Current methodologies require measurements with large specialized instruments in medical laboratories. There is a particular desire to have simplified and more sensitive approaches to measurement of FXa. The ability to easily quantify plasma FVIII and FIX on portable devices will broaden its use to home and point-of-care use.

### SUMMARY

This summary is provided to introduce a selection of concepts in a simplified form that are further described below in the Detailed Description. This summary is not meant or intended to identify or exclude key features or essential features of the claimed subject matter, nor is it intended to be used as an aid in determining the scope of the claimed subject matter.

According to one aspect, embodiments relate to fluorogenic peptide substrates and their linked solid-phase formats for the measurement of FXa. In some embodiments, the fluorogenic peptides allow for high sensitivity measurement of FXa, as generated from various hematological reactions. The specific sequences and composition of the fluorophores may allow the fluorophores to be utilized in both in-solution and solid-phase measurement formats in some embodiments. The approach may allow for greater sensitivity of measurement of FXa and measurement of upstream blood factors, including Factor VIII (FVIII) and Factor IX (FIX). In some embodiments, the composition of the peptides may include a C-terminus cleavable fluorophore, optional linker, optional attachment chemistry, and optional solid support.

The composition of the peptides may include an N-terminal group that contributes to the stability of the peptide, according to one aspect. In an embodiment, this may include using a carbamate (Z), succinate (Suc), benzyl carbamate (Bz), N-α-benzyloxycarbonyl (N-α-Cbz, or Z), or lysine (K). The recognition sequence may be DArg-Gly-Arg-Fluor, Glu(gamma-pip)-Gly-Arg-Fluor, or Glu(γ-OR)-Gly-Arg-Fluor where R═H, CH₃, or a combination of H and CH₃ a 50/50 mixture, in one embodiment. In one embodiment, additional sequence(s) may precede the core and desired recognition sequences. In some embodiments, these may include the use of Ile preceding GM. In some embodiments, FXa may cleave after the arginine residue in its cleavage site Ile-(Glu or Asp)-Gly-Arg. In some embodiments, the peptides here may have been further modified to optimized for sensitivity and specificity of FXa in biological reactions. Other combinations of sequences may be utilized in some embodiments as long as the core sequences are recognized, which give specificity to FXa measurements.

In one embodiment, the fluorophore may be linked to the C-terminus of the peptide recognition sequence via a cleavable peptide bond. Fluorophores fulfilling this requirement in some embodiments may include 7-Amino-4-9(trifluoromethyl)-coumarin (AFC), 7-amino-4-carbamoylmethyl-coumarin (ACC), and 7-amino-4-methylcoumarin (AMC). These fluorophores may be UV-excitable or excitable by a violet light source such as 405 nm LED or laser. Cleavage by FXa between the recognition sequence and the fluorophore releases the fluorophore in one embodiment, right-shifting the emission spectra so that the cleavage products may be measured by its fluorescence emission. The ACC fluorophore additionally has an amine group that may be functionalized and conjugation with an optional linker and attachment group for solid-phase reactions in one embodiment.

A linker between the ACC fluorophore and a solid support increases its distance from a surface, allowing it to have closer to in-solution phase enzyme kinetics in one embodiment. The longer the linker, the more distance may be between the FXa cleavage site and the solid support in one embodiment. A polyethylene glycol (PEG) linker or a carbon (C—C) linker may be utilized to define the distance in one embodiment. The linker may be conjugated to another linker or functionality to increase its length or change its attachment chemistry.

A plurality of attachment chemistries may be utilized for conjugation of the modified peptide to a solid support. This includes acrylate, COOH, amine, succinimidyl ester, biotin, —SH, -click, or a range of other possible attachment chemistries in some embodiments. The selection of attachment group is dependent in part based on a solid support that may be utilized for the reaction and also the available means of conjugation in some embodiments.

The solid support may be conventional microspheres, microplates, polystyrene plates, glass support surfaces, PEG microparticles, agarose beads, or other types of assay microparticles in some embodiments. The desired solid support is based on the desired instrument and readout. When using a plate reader, the assay can be implemented on a 96-well plate for ease-of-use in some embodiments. Glass support surfaces can be utilized along with microarray readers in some embodiments. PEG microparticles and microspheres can be utilized with a flow cytometry readout. PEG microparticles are porous, have low autofluorescence, and allow for easy coupling to various peptides in some embodiments. These attributes allow them to be highly suitable for solid-phase reactions with FXa in some embodiments.

Peptides using the ACC fluorophore can be readily incorporated into PEG microparticles in some embodiments. PEG microparticles with amine reactive groups, such as succinimidyl carboxymethyl ester (SCM), can be utilized to link a free amine group connected to the ACC fluorophore. The linking to a PEG molecule allows extension of the anchor point on the PEG molecule to the Gly-Arg cleavage sequence. Upon cleavage, the entire peptide will be released into solution in some embodiments. The ACC fluorophore remains anchored on the PEG microparticle in some embodiments. Due to the associated fluorescence emission right-shift, there is a detectable and, in some embodiments, significant increase in fluorescence at the cleaved fluorophore emission wavelength, leading to highly sensitive detection via flow cytometry or similar approach.

The use of fluorogenic peptides and fluorogenic peptides linked to solid supports has significant sensitivity advantages over absorbance-based approaches in some embodiments. Mildly hemophilic patients can be diagnosed and measured using the method without the need for two ranges, as is required in conventional kits. The increased sensitivity leads to greater accuracy and fewer reaction steps to obtain the answer in some embodiments. In one aspect, embodiments relate to a fluorogenic peptide substrate having the formula peptide-fluorophore-(linker)ₙ-(X)ₘ. The fluorogenic peptide substrate includes an attachment group, X, a peptide with a C-terminus, and a fluorophore cleavable at the C-terminus.

In one embodiment, the fluorophore is selected from the group including at least one of ACC, AMC, and AFC.

In one embodiment, the fluorophore of the fluorogenic peptide substrate is excitable by at least one of a 1N-light source and a violet light source.

In one embodiment, the fluorophore of the fluorogenic peptide substrate includes an amine group capable of being functionalized and conjugated with the at least one linker and the at least one attachment group, X.

In one embodiment, fluorogenic peptide substrate has the formula peptide-fluorophore-(linker)ₙ-(X)ₘ, n is an integer from 0 to 4 and m is an integer from 0 to 4.

In one embodiment, the C-terminus of the fluorogenic peptide substrate is Arg.

In one embodiment, fluorogenic peptide substrate includes the sequence DArg-Gly-Arg.

In one embodiment, the fluorogenic peptide substrate includes the sequence Ile-Glu(gamma-pip)-Gly-Arg.

In one embodiment, the fluorogenic peptide substrate includes the sequence Ile-Glu(gamma-OR) and R is selected from the group including at least one of H and CH₃.

In one embodiment, the linker of the fluorogenic peptide substrate is selected from the group including PEG and C—C.

In one embodiment, the linker of the fluorogenic peptide substrate is spherical PEG synthesized generating microfluidic droplets.

In one embodiment, the fluorogenic peptide substrate linker is rectangular PEG synthesized by stop-flow lithography.

In one embodiment, the fluorophore of the fluorogenic peptide substrate is configured to be cleaved at the C-terminus by FXa.

In one embodiment, upon cleavage at the C-terminus, the fluorophore is configured to remain bound to the linker.

In one embodiment, the attachment group X of the fluorogenic peptide substrate includes at least one of —NH₂, —COOH, —SH, -SCM, -acrylate, -click, maleimide, -alkyne, -ITC, —NHS, -SMCC, -ALD, -EPOX, -ester, hydrazide, -SIL, and -VA.

In one embodiment, the peptide of the fluorogenic peptide substrate includes an N-terminus.

In one embodiment, the N-terminus of the fluorogenic peptide substrate includes at least one of Z, Suc, Lys, Bz, and Cbz group.

In another aspect, embodiments relate to a method of measuring enzymatic activity. The method includes using at least one of the peptide of a fluorogenic peptide substrate and the fluorogenic peptide substrate to detect at least one protein in a clotting cascade comprising FXa.

In one embodiment, the protein uses in the method is selected from the group including at least one of FXa, FVIII, and FIX.

In one embodiment, the method is used to detect the at least one protein in a patient with hemophilia.

### BRIEF DESCRIPTION OF DRAWINGS

FIG. 1 shows the conceptual diagram of some embodiments. There is the peptide sequence, linked at the C-terminus to the cleavable fluorophore, optionally attached to a linker and/or an attachment group that allows for attachment to a solid support.

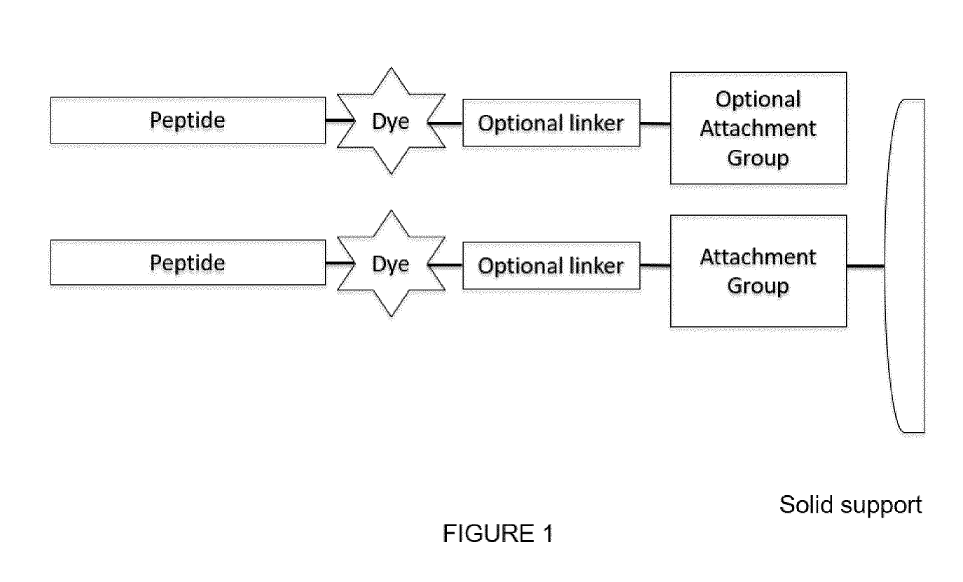

FIG. 2 shows an example of some types of solid supports that can be utilized with some embodiments. It includes spherical and rectangular PEG microparticles and also microspheres.

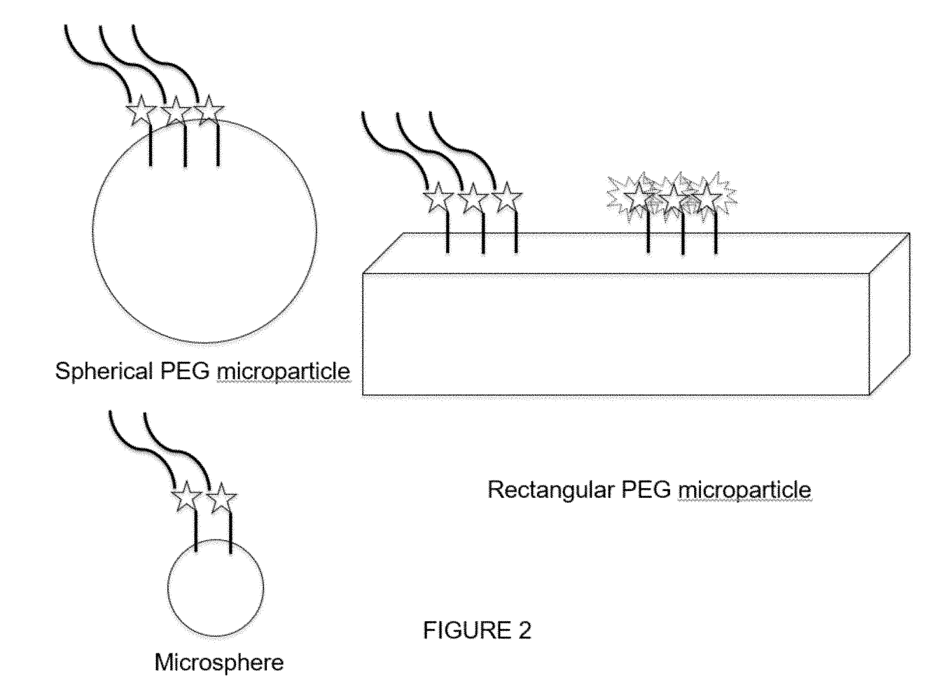

FIG. 3 shows cleavage of the peptides in solution and linked to a solid support. Cleavage on the solid support leaves the fluorophore on the support, thus allowing for fluorescence measurements on the solid support.

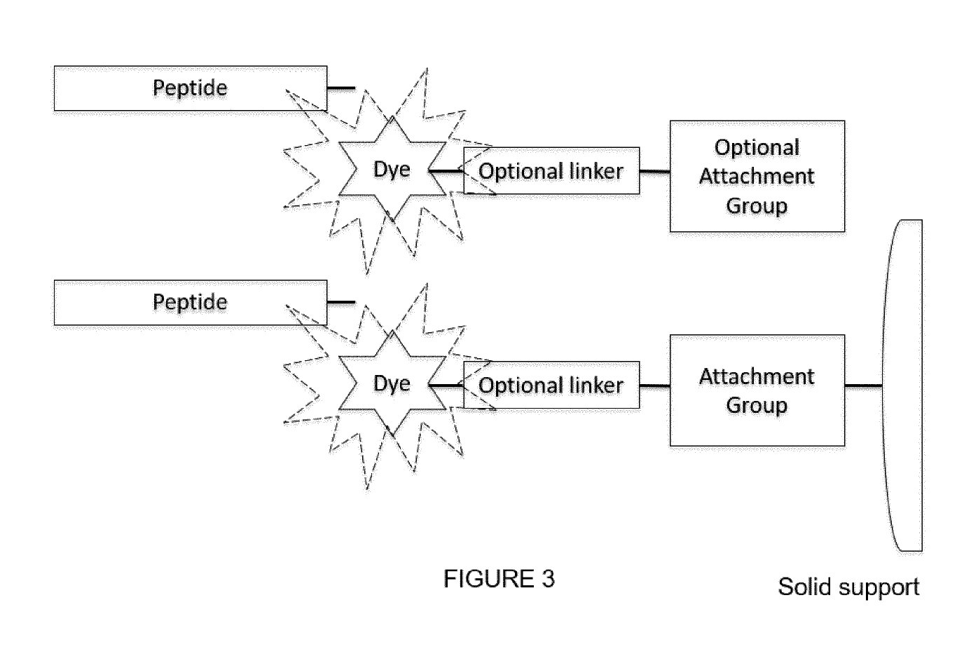

FIG. 4 shows some of the peptide sequences described in some embodiments. The peptides show an N-terminal group for increasing the stability of the peptide, the cleavage sequence, the fluorophore (selected from -AMC, -ACC, and -AFC), linkers, and also the attachment chemistry. The peptides in this figure are examples and other combinations are possible based on the information provided.

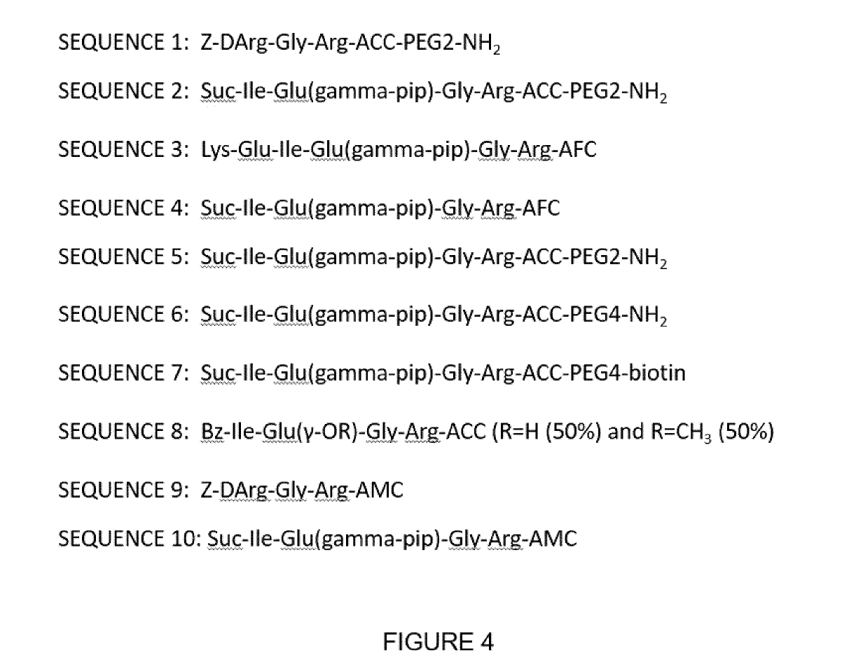

FIG. 5 shows a partial list of attachment chemistries and also linkers.

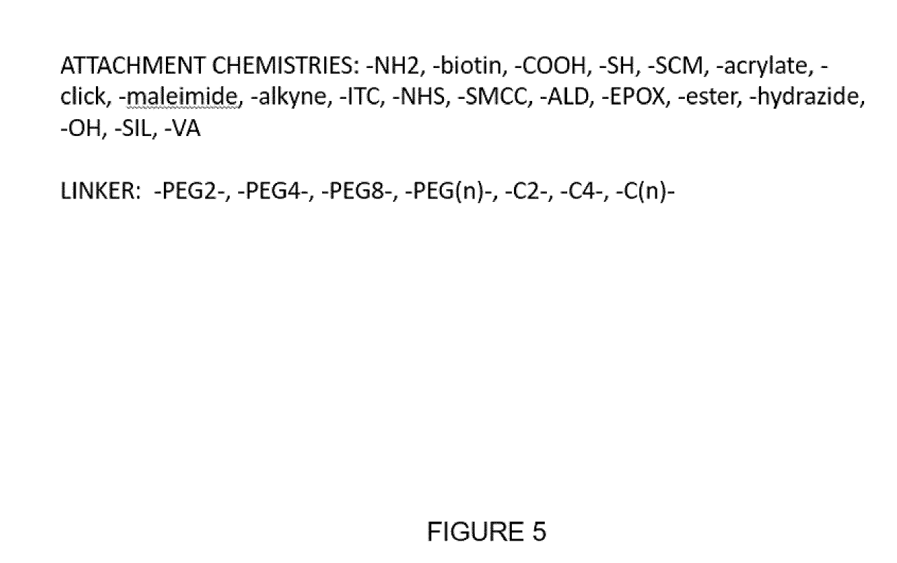

FIG. 6 shows the structures of the three fluorophores utilized in some embodiments.

FIG. 7 shows the chemical structures for Suc-Ile-Glu(gamma-pip)-Gly-Arg-Acc-Peg2-NH₂ and Suc-Ile-Glu(gamma-pip)-Gly-Arg-Acc-Peg4-NH₂.

FIG. 8 shows the chemical structure for Z-D-Arg-Gly-Arg-ACC-PEG4-NH₂.

FIG. 9 shows the chemical structure for Z-D-Arg-Gly-Arg-ACC-PEG₂-NH₂.

FIG. 10 shows the chemical structure for Z-D-Arg-Gly-Arg-ACC-PEG(n)-biotin.

FIG. 11 shows the excitation and emission spectra for 7-amino-4-trifluoromethylcoumarin and 7-amino-4-methylcoumarin.

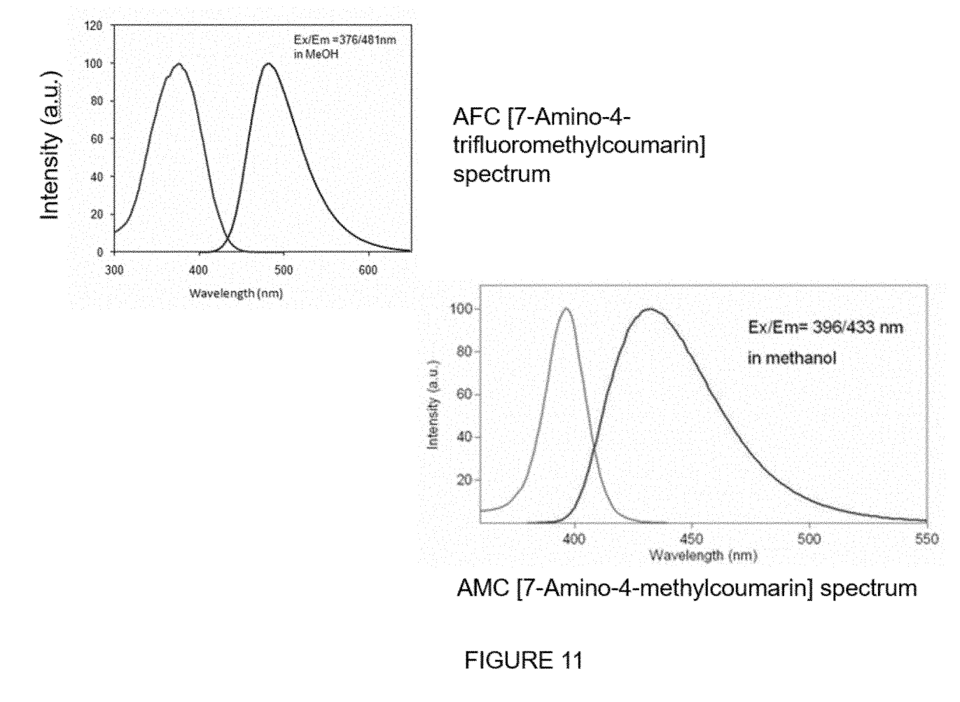

FIG. 12 shows the excitation and emission spectra for ACC and AFC.

FIG. 13 shows the chromogenic pNA and ACC peptides' sensitivity to varying FXa concentrations 3900 ng/mL, 390 ng/mL, 39 ng/mL, 3.9 ng/mL, 0.39 ng/mL, and 0, respectively. A base 10 log scale is used on the x-axis. Graph (a) shows the limit of detection of pNA peptide is between 0.39 and 3.9 ng/mL, (P<0.0210). Graph (b) shows the limit of detection of Z-ACC is between 0 and 0.39 ng/mL (P<0.0004).

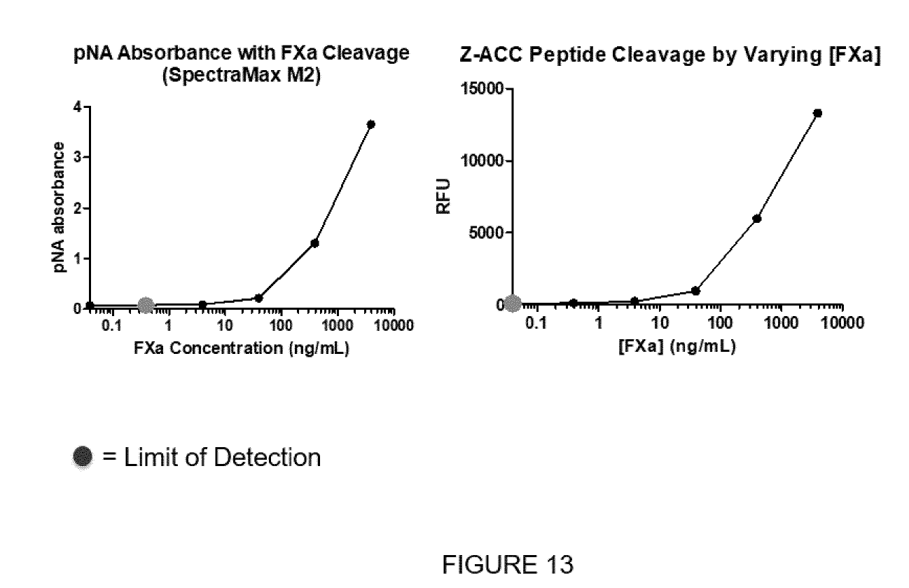

FIG. 14 shows the Z-ACC-linked microparticles response to varying FXa concentrations detected using miniature flow cytometer. The Z-ACC peptide microparticles were reacted with varying FXa concentrations (3900 ng/mL, 390 ng/mL, 39 ng/mL, 3.9 ng/mL, 0.39 ng/mL and 0). An increase of signal intensity was observed with increasing FXa concentration. All points are statistically different. 0 and 0.39 ng/mL are significant with P<0.0001.

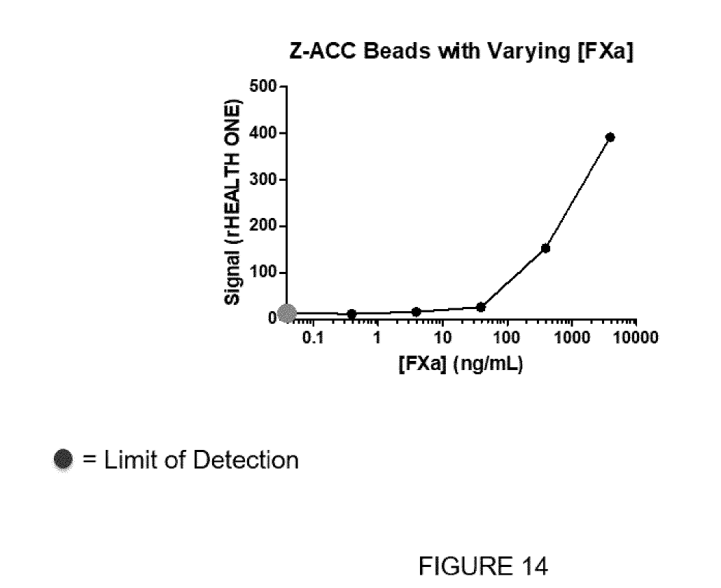

### DETAILED DESCRIPTION

The desired peptides and peptides linked to microparticles have specific cleavage sequences for FXa. In particular, they all contain a cleavable fluorophore at the C-terminus selected from AMC, ACC, or AFC. Cleavage of the peptide bond between the terminal amino acid and the fluorophore releases the fluorophore, leading to a right shifting of the spectra and increase in detectable fluorescence.

The ACC fluorophore is bifunctional and may be utilized when a solid support is utilized in some embodiments. Cleavage of the substrate allows the ACC fluorophore to remain on the solid support, allowing for detection along with the solid support. This is particularly important with a microparticle-based flow assay, which allows for measurement of concentrated ACC fluorophore on the microparticles.

The ACC fluorophore may be utilized when high sensitivity and assay dynamic range are desired. In particular, the ACC fluorophore has minimal spectral overlap between the uncleaved and cleaved states at the emission maxima (460 nm) of the fluorophore. This lack of a spectral tail for the uncleaved fluorophore make it such that, in some embodiments, there may be a large difference between the uncleaved and cleaved substrates. This is particularly important when a broad FXa assay range with high sensitivity is desired.

With the use of the ACC fluorophore on solid support, in some embodiments, a linker is also present. In some embodiments, the linker may be made of PEG that is spaced of two to four PEG molecules long. This will allow it to maintain functionality in enzymatic cleavage processes by offering a set distance between the peptide and the solid support. In some embodiments, the ACC fluorophore is further conjugated to another PEG molecule that increases the linker length on the solid support even further. C—C linkers can also be utilized as well or other PEG spacer lengths. The synthesized peptide may be attached to a PEG molecule with a molecular weight (MW) that may be greater than 1 kilodaltons (kD) in some embodiments. The solid support may further be made of PEG to minimize non-specific binding interactions and to decrease autofluorescence in some embodiments.

The attachment chemistry on the peptide may be a molecule that is easily conjugated. It is, in some embodiments, selected from the following list: —NH₂, —COOH, —SH, -SCM, -acrylate, -click, maleimide, -alkyne, -ITC, —NHS, -SMCC, -ALD, -EPOX, -ester, hydrazide, —OH, -SIL, -VA. Easy to synthesize chemistry is best in some embodiments. For instance, the amine —NH₂ functionality is readily added during peptide synthesis, in some embodiments. Amine groups may be linked to succinimidyl chemistries readily.

One of the sequences is DArg-Gly-Arg preceding the fluorophore in some embodiments. Another sequence is Ile-Glu(gamma-pip)-Gly-Arg preceding the fluorophore in some embodiments. Other embodiments utilize the sequence Ile-Glu(gamma-OR) where R═H or R═CH₃ or where R is a 50:50 mixture of —H or —CH₃. Some of the sequences, such as DArg-Gly-Arg, Ile-Glu(gamma-pip)-Gly-Arg, and Ile-Glu(gamma-OR) allow for maximal sensitivity and specificity to FXa.

For stability, in some embodiments, the N-terminus may have the equivalent of a capping group, which may include the Z, Suc, Lys, Bz, or Cbz groups. In this manner, the peptide may be protected from degradation and may have the optimal stability in biological reactions. Other N-terminal protecting or capping groups may be utilized as well and, in some embodiments the approach is consistent with existing peptide synthesis methods.

In some embodiments, the solid support may be made from polymerized. PEG microparticles. In some embodiments, PEG microparticles have low autofluorescence, are porous, have low non-specific binding, and can have different functionalities. In particular, PEG with acrylate groups may be utilized to form hydrogel particles using ultraviolet (UV) exposure. PEG microparticles thus have desirable attributes for biological applications.

Peptides can be readily incorporated into these hydrogels with the use of bifunctional linkers such as ACRYL-PEG-SCM functionality. The amine functionalized the peptide can be reacted with the -SCM group and then incorporated into the polymerization mixture. This requires reacting at pH>8, typically with the use of sodium bicarbonate at 0.1M, freeing up the electron pair on the amine group for the reaction.

PEG microparticles can be spherical or rectangular. Spherical microparticles may be generated through the generation of microfluidic droplets. This approach utilizes a microfluidic device that is fabricated with polydimethylsiloxane (PDMS). The PDMS device is fabricated utilizing an SU-8 master mold. The microfluidic device may be fabricated using replica molding. A mixture of PDMS prepolymer and curing agent (10:1, Sylgard 184, Dow Corning Co) is mixed, degassed and poured onto the SU-8 master and cured at 65° C. Droplets are formed using a droplet junction and then polymerized using UV light in the channel with a photoinitiator added to the mixture.

Rectangular microparticles may be synthesized by stop-flow lithography. In this approach, a PDMS channel is utilized and synchronized valves and shutters control passage of the PEG prepolymer mixture into the chip. UV light exposure polymerizes the microparticles through a photomask placed in the field stop position of the illuminating path. Slide-based polymerization may also be utilized to polymerize rectangular microparticles or particles of different shapes. This is done by allowing UV light to go through a photomask to pattern a PEG prepolymer mixture. The polymerized microparticles may be washed and utilized for assays.

Performance testing of the peptides may be done using purified FXa and plotting Michaelis-Menten curves and comparing the turnover rate (kcat). This reaction may be done at 37° C. and monitored over time for cleavage rate. A spectrophotometer, such as the SpectraMax M2 in kinetic mode may be utilized with excitation at 405 nm and emission at 450 nm.

The peptide substrates and peptides immobilized on a solid support may also be utilized for testing in FVIII or FIX assays. This includes taking a plasma sample, diluting it in a buffer such as 10 mM Tris with 01% BSA and then mixing it with FIXa, PL, and Ca2+ to form the tenase complex. The tenase complex then cleaves FX to make FXa, which then cleaves the substrate to give rise to fluorescence that is measured and is directly proportional to the plasma's FVIII level in some embodiments. PEG-based microparticles may be read out using a flow cytometer or micro-flow cytometer. In solution measurements may be done via a spectrofluorometer such as the SpectraMax M2.

Some embodiments may be applied to the measurement of FXa, FVIII, FIX, and other proteins in the clotting cascade that involves FXa. The fluorogenic nature of the reaction may allow for high sensitivity and dynamic range, which is particularly applicable for measurement of hemophilia patient in various settings and low levels of blood factors in some embodiments. The compatibility with solid phase reactions, may allow it to be utilized in a broad range of assay formats for measurements of various blood factors. Often times in diagnostic assays, hemolysis is an issue. Traditional chromogenic substrates, such as pNA, absorb at the same wavelength as hemoglobin. The fluorogenic substrates described here are less susceptible to hemolysis.

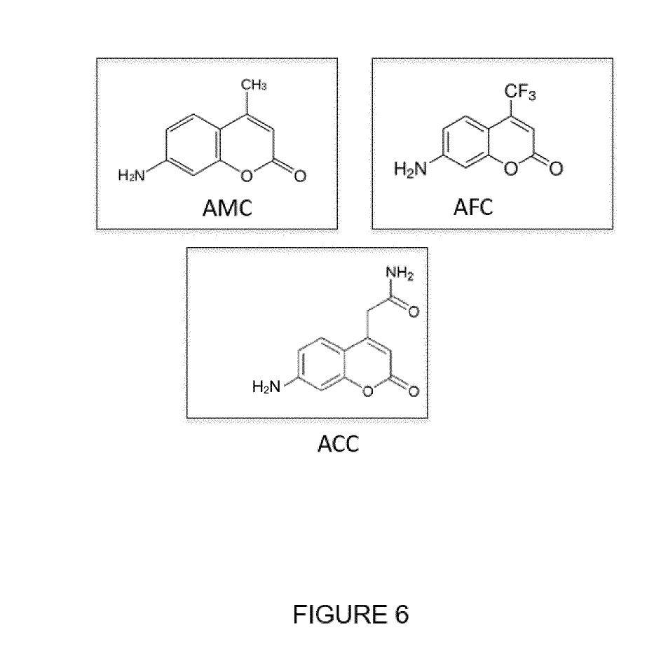

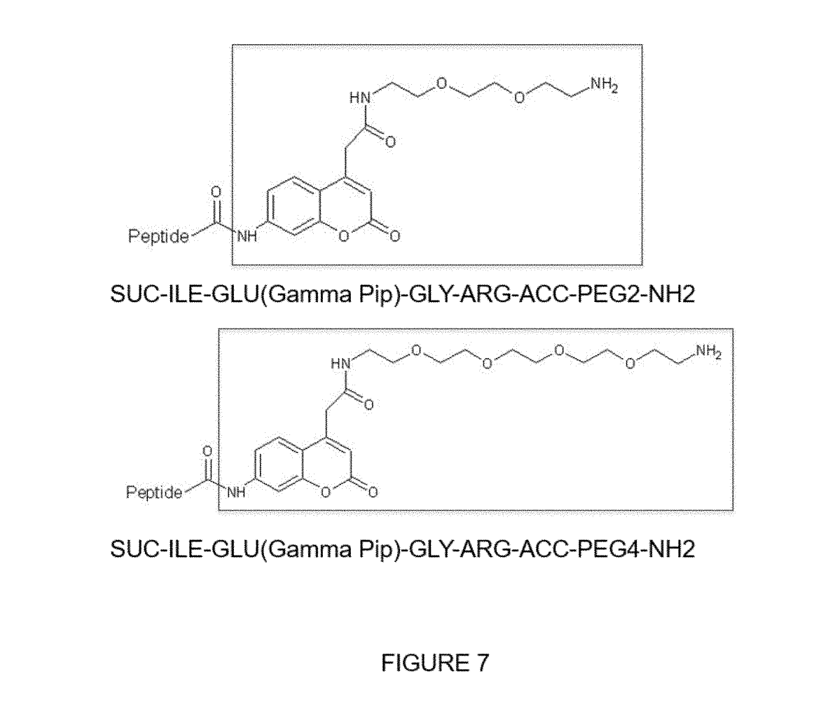

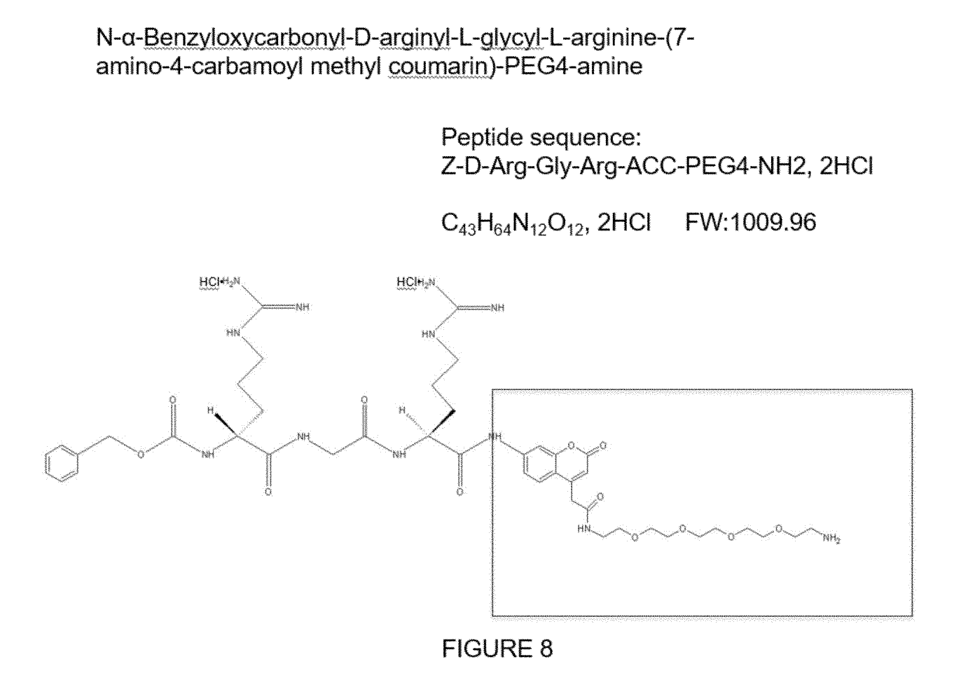

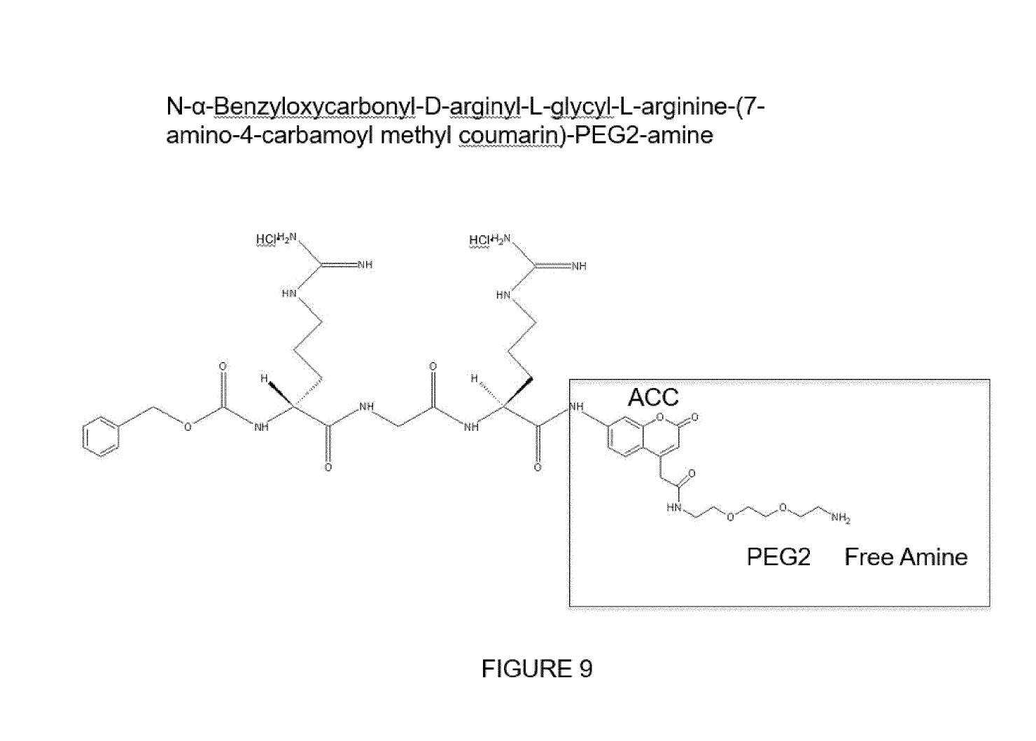

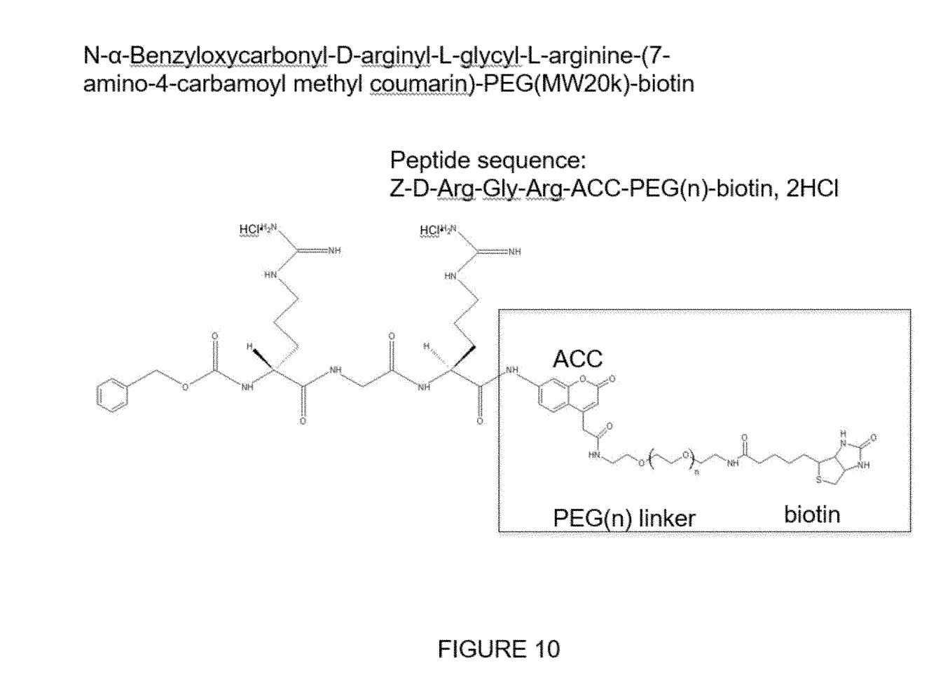

FIGS. 6-10 highlight the cleavable fluorophore, linkers, and attachment groups in accordance with some embodiments.

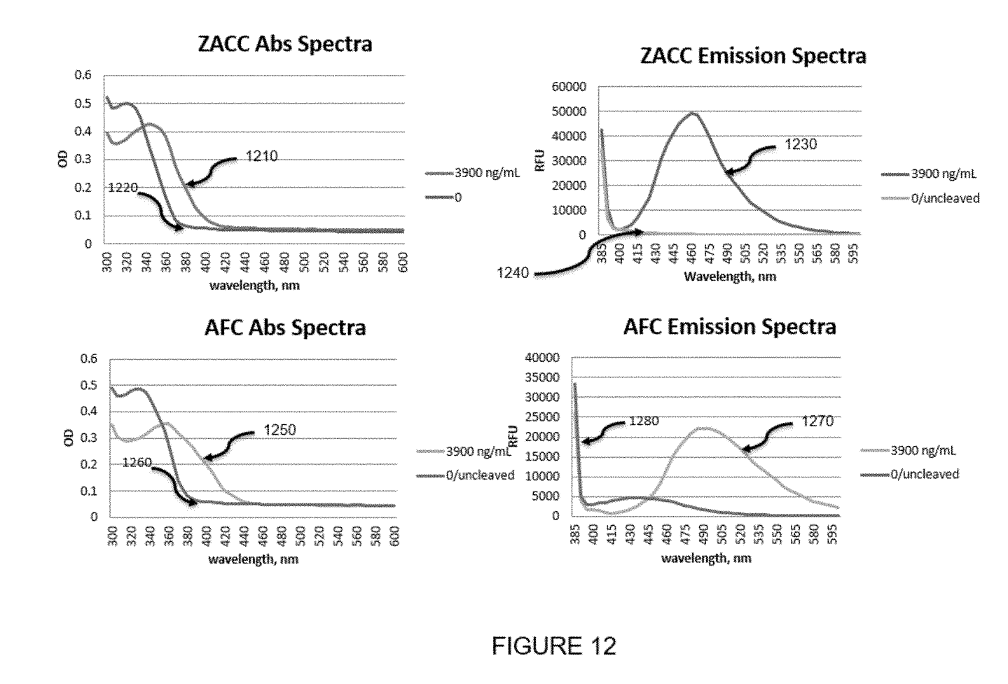

FIG. 12 shows the excitation and emission spectra for ACC and AFC. The ACC absorption spectra at a concentration of 3900 ng/mL 1210 is compared to the control 1220. The ACC emission spectra at a concentration of 3900 ng/mL 1230 is compared to the control 1240. The AFC absorption spectra at a concentration of 3900 ng/mL 1250 is compared to the control 1260. The AFC emission spectra at a concentration of 3900 ng/mL 1270 is compared to the control 1280.

Those skilled in the art will recognize or be able to ascertain using no more than routine experimentation and/or engineering, many equivalents to the specific embodiments of the invention described herein. The scope of the present invention is not intended to be limited to the above Description, but rather is as set forth in the claims that follow the reference list.

What is claimed is:

1. A fluorogenic peptide substrate having the formula
peptide-fluorophore-(linker)n-(X)m
wherein
X is an attachment group;
the peptide comprises a C-terminus; and
the fluorophore is cleavable at the C-terminus.

2. The fluorogenic peptide substrate of claim 1 wherein the fluorophore is selected from the group consisting of ACC, AMC, and AFC.

3. The fluorogenic peptide substrate of claim 1 wherein the fluorophore is excitable by at least one of a UV-light source and a violet light source.

4. The fluorogenic peptide substrate of claim 1 wherein the fluorophore comprises an amine group capable of being functionalized and conjugated with the at least one linker and the at least one attachment group, X.

5. The fluorogenic peptide substrate of claim 1 wherein:
n is an integer from 0 to 4; and
m is an integer from 0 to 4.

6. The fluorogenic peptide substrate of claim 1 wherein the C-terminus is Arg.

7. The fluorogenic peptide substrate of claim 1 wherein the peptide comprises the sequence DArg-Gly-Arg.

8. The fluorogenic peptide substrate of claim 1 wherein the peptide comprises the sequence Ile-Glu(gamma-pip)-Gly-Arg.

9. The fluorogenic peptide substrate of claim 1 wherein:
the peptide comprises the sequence Ile-Glu(gamma-OR);
and R is selected from the group consisting of H and CH₃.

10. The fluorogenic peptide substrate of claim 1 wherein the linker is selected from the group comprising PEG and C—C.

11. The fluorogenic peptide substrate of claim 1 wherein the linker is spherical PEG synthesized generating microfluidic droplets.

12. The fluorogenic peptide substrate of claim 1 wherein the linker is rectangular PEG synthesized by stop-flow lithography.

13. The fluorogenic peptide substrate of claim 1 wherein the fluorophore is configured to be cleaved at the C-terminus by FXa.

14. The fluorogenic peptide substrate of claim 1 wherein, upon cleavage at the C-terminus, the fluorophore is configured to remain bound to the linker.

15. The fluorogenic peptide substrate of claim 1 wherein the attachment group X comprises at least one of —NH₂, -biotin, —COOH, —SH, —CM, -acrylate, -click, maleimide, -alkyne, -ITC, —NHS, -SMCC, -ALD, -EPOX, -ester, -hydrazide, —OH, -SIL, and -VA.

16. The fluorogenic peptide substrate of claim 1 wherein the peptide comprises an N-terminus.

17. The fluorogenic peptide substrate of claim 16 wherein the N-terminus comprises at least one of Z, Suc, Lys, Bz, and Cbz group.

18. A method of measuring enzymatic activity comprising using at least one of the peptide of the fluorogenic peptide substrate of claim 1 and the fluorogenic peptide substrate of claim 1 to detect at least one protein in a clotting cascade comprising FXa.

19. The method of claim 18 wherein the at least one protein comprises FXa, FVIII, and FIX.

20. The method of claim 18 wherein the method is used to detect the at least one protein in a patient with hemophilia.
